(19) United States
(12) Patent Application Publication
(10) Pub. No.: US 2013/0330425 A1
(43) Pub. Date: Dec. 12, 2013

(54) Plant retinol formulations and associated method

(71) Applicant: Le Thuy Vuong, Petaluma, CA (US)

(72) Inventor: Le Thuy Vuong, Petaluma, CA (US)

(21) Appl. No.: 13/897,303

(22) Filed: May 17, 2013

Related U.S. Application Data
(60) Provisional application No. 61/688,563, filed on May 17, 2012.

(51) Int. Cl. A61K 36/185

(52) U.S. Cl. CPC A61K 36/185
USPC 424/725

(57) ABSTRACT

Nutritional formulations and methods to produce safe vitamin A/retinol are described. In particular, methods and formulations containing plant pro-vitamin A carotenoids from momordica cochinchinensis spreng are disclosed. The formulations and associated method are characterized by high concentration of provitaminA carotenoids in a water soluble form with enhanced stability and absorption. This invention describes methods to produce water-soluble provitamin A powder from “gac” and formulas to enhance absorption of plant provitamin A carotenoids to a subject. The method described herein requires no chemical solvents, nor involves genetic modification, also delivers other nutrients to improve nutritional status of subjects. This method provides a safe source of vitamin A to be used in pharmaceuticals, cosmeceuticals, nutraceuticals and food supplements for humans and animals.

### CROSS REFERENCE TO RELATED APPLICATION

This application claims priority from U.S. provisional patent No. 61/688,563 filed May 18, 2012, the contents of which are to be taken as incorporated herein by this reference.

### FIELD OF INVENTION

This invention relates to formulations and methods to enhance absorption of provitamin-A carotenoids. Accordingly, the invention involves the fields of botany, nutritional, skin care, cosmetics, nutraceuticals, and natural ingredients.

### OBJECTS AND ADVANTAGES

Plant retinol in the form of provitamin A carotenoids provide a safe and natural way to prevent vitamin A deficiency disorders and to improve health of at risk populations. However, bioavailability of provitamin A carotenoids is critical in order to effectively use them to impart health benefits. The objectives and hence advantages of this invention is to enhance absorption and conversion of provitamin A carotenoids by incorporating effective amount of momordica cochinchinenis spreng. extract to beverage, food, foodstuff, or supplement formulations to enhance absorption of provitamin A. This invention is an improvement to the method of extracting carotenoids from momordica cochinchinensis. The formulations can be applied to programs to prevent vitamin A deficiency among children and pregnant women without incurring toxicity related to high dose retinol or retinoic acid.

### BRIEF SUMMARY OF THE INVENTION

Retinol or vitamin A is important for human health. However, large doses of retinol are toxic. Beta-carotene from plants is converted to retinol in humans. High doses of dietary beta-carotene are not toxic. The accumulation of beta-carotene in fat cells in long-term intake of large doses of beta-carotene, which turns skin yellow in a condition called beta-carotenosis does not cause any systemic damage. Stored beta-carotene can be used when liver retinol is depleted. In addition to providing vitamin A, beta-carotene is an anti-oxidant, and thus plays an important role in cancer prevention. Vitamin A, as well as carotenoids are lipophylic, and unsoluble in water. Efficient absorption of provitamin A carotenoids requires sufficient amount of fat and protein. Bioavailability and stability of provitamin A carotenoids also are improved in a lipid matrix. Momordica cochinchinensis Spreng, also known as “gac” contains high levels of lycopene and beta-carotene. Methods of extracting an oil rich in carotenoids from “gac” has been discussed. This invention describes methods to produce water-soluble provitamin A powder from “gac” and formulas to enhance absorption of plant provitamin A to a subject. The method requires no chemical solvents, nor involves genetic modification, also delivers other nutrients to improve nutritional status of subjects.

### BACKGROUND OF THE INVENTION

Retinol also known as vitamin A was discovered over almost a century ago. Retinol occurs in ester form in liver, egg yolk, butter, cheese, cream, oily fish (salmon), and in vitamin A fortified food, or as provitamin A-carotnoid in green-yellow vegetables, and in food artificially colored with beta-carotene. Allowances of vitamin A is 500 mcg to 1 mg/day (depending on weight). Vitamin A is essential factor for normal vision, also has important roles in immune system, growth, reproduction, skin health, and in the prevention of specific cancers.

In many populations, vitamin A deficiency is still a problem, which leads to growth stunting, compromised immune system, infections, xerophthamia, and blindness. Vitamin A (performed) is found almost exclusively in animal products, such as meats, liver, fish liver oils, egg, milk and dairy products, and fortify processed foods. Because vitamin A is fat soluble and can be stored, primarily in the liver, routine consumption of large amounts of vitamin A over a period of time can result in toxic symptoms, including liver damage, bone abnormalities and joint pain, alopecia, headaches and vomiting, and skin desquamation. Hyperylaminosis A appears to be due to abnormal transport and distribution of vitamin A and retinoids caused by overloading of the plasma transport mechanisms (Smith, F. R & Goohdman, D. S. 1976). Very high single doses can also cause transient acute toxic symptoms that may include bulging fontanels in infants (Humphrey. J. H. 1998., de Francisco. A. 1995).

Vitamin A excess has been reported of congenital defects in the progeny of women who had ingested large quantities of vitamin A early in pregnancy (Pilotti and Scorta, 1965; Bernhardt and Dorsey, 1974; Mounoud et al., 1975; Rosa, 1984). A recent reports of malformations of infants of pregnant women taking high doses of vitamin A analogs for treatment of acne (Stange et al., 1978; Rosa, 1983, 1984; Lamme et al., 1985). The intake of large doses of vitamin A, therefore, should be avoided by women who could conceivably be pregnant, and supplementation even with modest does should be undertaken with caution (Underwood, 1986).

Pro-vitamin A carotenoids, such as beta-carotene can be converted into retinol in human's body, provides a safe source of vitamin A. Beta-carotene is found in green leafy vegetables and fruits. Carotenoids provide ⅔ of the vitamin A activity in the diets of economically deprived populations (FAO Corporate Documentary Repository). ProvitaminA carotenoids are found in many fruits and vegetables such as carrots, spinach, and sweet potatoes. One food which is extremely high in beta-carotene is momordica cochinchinensis Spreng., also known as “gac” fruit. Methods to extract nutrients from the “gac” fruit, and different usages have been published. Other sources of beta-carotene include red palm oil from Indonesia, and bruiti from Brazil. Carrots have been well known as the main source of dietary beta-carotene in western diets. In order for absorption and conversion efficiency of pro-vitamin A, dietary fat and protein should be presence. Dietary restriction in energy, proteins, and some other micronutrients can limit hepatic synthesis of proteins specific to mobilization and transport of vitamin A. Low dietary intake causes mobilization of stored carotenoids from fatty tissues throughout the body (Blomhoff, R. 1991).

Red, orange, yellow fruits and tubers are good sources of carotenoids, such as “gac” fruit, tomatoes (USDA ref.), mangoes. Carotenoids in those fruits include lycopene (tomatoes), alpha and beta-carotene, lutein, canthxanthin. Among those carotenoids, only beta-carotene, alpha carotene can be converted into vitamin A. (Lakshman and Okoh, 1993). In fact beta-carotene is the major dietary pre-cursor of vitamin A. This carotenoid is synthesized in plants by the pathway that synthesizes a precursor of carotenoids, lycopene. Methods using genetic engineering to produce a high beta-carotene tomatoes have been published (Rosati et al., 2000, Roemer et al., 2000, Giuliano et al., 2000). In one of those methods, bacterial phytoene desaturase capable of transforming phytoene into lycopene is fused to a plastidic transit peptide and introduced to the tomato plants. This method unexpected resulted in threefold increase in beta-carotene (but not lycopene) and decrease in total carotenoids. Another method, modified gene expression to up-regulated ripening of tomatoes at the same time suppresses the competing synthesis of lycopene by introducing Arabidopsis into tomatoes gene.

Since plant beta-carotene, a precursor of vitamin A (provitamin A), supplementation of the diet with beta-carotene can prevent vitamin A deficiency disorders (VAD) and improve overall health. Methods and formulations to enhance absorption and bioconversion of provitamin A from plant to benefit populations at risk of VAD continues to be sought.

### BRIEF DESCRIPTION OF DRAWINGS

FIG. 1: Plasma response graphs showing provitamin A of a subject after one dose of enhanced absorption vitamin A formulation 1 (momordica) as compared to beta-carotene capsule (marketed as food supplement).

### DETAILED DESCRIPTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting.

### DEFINITION OF TERMS

In describing and claiming the present invention, the following terminology will be used.

The singular forms “a,” “an,” and “the” include plural referents unless the context clearly dictates otherwise. Thus, for example, reference to “a carrier” includes reference to one or more of such carriers, and reference to “an excipient” includes reference to one or more of such excipients.

As used herein, “formulation” and “composition” may be used interchangeably herein, and refer to a combination of two or more elements, or substances. In some embodiments a composition may include an active agent and a carrier.

As used herein, “effective amount” or “antioxidant absorption enhancing amount” refers to an amount of an ingredient, namely composition, is sufficient to achieve an intended compositional or physiological effect. Thus, a “therapeutically effective amount” refers to a non-toxic, but sufficient amount of an agent, including active agents, to achieve therapeutic results in preventing or ameliorating a condition for which the active agent is known to be effective, or in attaining a desired physiologic effect. It is understood that various biological factors may affect the ability of a substance to perform its intended task. Therefore, an “effective amount,” “antioxidant absorption enhancing amount,” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “carrier” or “inert carrier” refers to a substance with which a bioactive agent or a nutritional agent may be combined to achieve a specific dosage formulation for delivery to a subject. As a general principle, carriers must not react with the bioactive agent in a manner which substantially degrades or otherwise adversely affects the bioactive agent or its potency.

As used herein, “excipient” refers to substantially inert substance which may be combined with an active agent and a carrier to achieve a specific dosage formulation for delivery to a subject, or to provide a dosage form with specific performance properties. For example, excipients may include but are not limited to binders, lubricants, etc., but specifically exclude active agents and carriers.

As used herein, “subject” refers to a mammal that may benefit from the administration of a composition or method as recited herein. Most often, the subject will be a human.

As used herein, “administration,” and “administering” refer to the manner in which an active agent, or composition containing such, is presented to a subject. Administration can be accomplished by various routes well-known in the art including oral and non-oral methods.

As used herein, “coadministration” refers to administration of two or more active agents in a manner that will allow them to be present together in-vivo for period of time. Accordingly, while the term “coadministration” includes simultaneous administration of two or more active agents, and administration from a single formulation, it is to be understood that it is not limited thereto.

“Oral administration” can be achieved by swallowing, chewing, or sucking of an oral dosage form comprising the drug. Examples of well known oral dosage forms include tablets, capsules, caplets, powders, granulates, beverages, syrups, elixirs, confections, or other food items, etc.

As used herein, “extract” when used in connection with a plant, tree, herb, fungus, etc., refers to material which has been removed from the source, or a portion thereof, including the flower, fruit, seed, peel, leaf, root, bark, stem, etc. As will be recognized by those of ordinary skill in the art, extracts may be either crude or refined to a selected degree in order to isolate specified materials or active agents. Extracts can take a variety of forms including powders, juices, purees, etc. A number of extraction processes that can be employed to produce the compositions of various types will be recognized by those skilled in the art, such as dehydration, lyophilization, etc.

“Synergistic”, “synergism”, “synergistically effective” or “synergistically enhances”, may be used interchangeably and refer to a situation in which the combined effect of two agents is greater than which would be predicted from their individual effects. Various mechanisms for calculating or otherwise determining synergism are known to those of ordinary skill in the art.

“Antioxidant” refers to a chemical compound, an enzyme or other organic molecule which prevents free radicals from causing oxidation of molecules in the body. Susceptible molecules include without limitation, such vital entities as DNA, RNA, lipids (fats), and proteins. The antioxidant, by reacting with the oxidant, protects these important molecules from being damaged. Examples of antioxidants include without limitation, vitamins A, C, E, carotenoids, polyphenols, and certain minerals.

The term “Gac Fruit,” “Gac Extract,” “Gac fruit extract,” “Gac plant,” and “Gac Fruit Puree” refer to a product made from the fruit of all strains and hybrids of the plant Momordica cochinchinensis Spreng, or of plants significantly related thereto, grown anywhere in the world including blends, mixtures, and combinations of such strains and relatives.

As used herein, the term “conventional nutritional supplement” refers to antioxidant supplements in any form which do not contain Gac fruit or a Gac fruit extract.

As used herein, the terms “bioavailable” or “bioavailability” when used in connection with antioxidants refer to the portion of a formulation which is absorbed or taken up into a subject's general circulation. The term “enhanced bioavailability” refers to an increase in the percentage of administered nutrient which is absorbed by the subject into general circulation. Within the context of this document, the nutrients are provitamin A carotenoids, and/or vitamin A. Bioavailability and bio-conversion efficiency of provitamin A carotenoids are quantified by measuring plasma beta-carotene and retinol increase before and after feeding. Carotenoids separation were done on a reverse-phase HPLC system following published method (Liquid chromatography-mass spectrometry method for the quantification of bioavailability and bioconversion of beta-carotene to retinol in humans. Wang Y et al. Anal Chem. 2000 Oct. 15; 72(20):4999-5003.).

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of 0.5 to 400 should be interpreted to include not only the explicitly recited concentration limits of 0.5 and 400, but also to include individual concentrations within that range, such as 0.5, 0.7, 1.0, 5.2, 8.4, 11.6, 14.2, 100, 200, 300, and sub-ranges such as 0.5-2.5, 4.8-7.2, 6-14.9, 55, 85, 100-200, 117, 175, 200-300, 225, 250, breadth of the range or the characteristic being described.

### The Invention

This invention is an improvement of the method to extract beta-carotene from the fruit momordica cochinchinensis spreng, also known as “gac” by Vuong (U.S. Pat. No. 6,770,585). This fruit has been recognized to contain highest concentrations of carotenoids, mainly lycopene and beta-carotene. It is of interest to improve the absorption of provitamin A carotenoids from available sources without the use of any chemical solvents, nor genetic modification.

The present invention encompasses methods and nutritional formulations for imparting positive health and nutritional benefits to a subject. In one aspect, such a nutritional formulation includes the disbursement of momordica cochinchinensis powder (mc powder) in a protein solution. In another aspect, such a nutritional formulation includes the disbursement of mc powder in a starch solution. In another aspect, such a nutritional formulation includes the mixture of mc oil extract with a rice meal. In another aspect, a method to improve absorption of provitamin A in a subject can include administering the nutritional formulation which includes mc powder in combination with at least one other provitamin A source to the subject. In another aspect of the present invention a nutritional formulation is provided, wherein the active ingredients, mc extract is co-administered with one or more provitamin A fruits and vegetables.

As noted above, beta-carotene, a precursor of vitamin A can be found in many yellow and red fruits and vegetables, such as carrots, mangoes, tomatoes, spinach. Vitamin A is essential for immune system, integrity of epithelial cells, eye sight and an effective prevention from degenerative diseases including some types of cancer. Plant vitamin A is a safe and abundant source for retinol. Enhancement of absorption at the same time deliver other nutrients to mobilize and store vitamin A can benefit populations at risk of vitamin A deficiency.

As will be recognized by those of ordinary skill in the art, provitamin A carotenoids sources are abundant in nature and can be derived or extracted from many different species of plants. Specific examples of provitamin A sources that can be utilized in the present invention include without limitation, fruits and vegetables.

The nutritional formulation of the present invention can increase vitamin A levels in a subject after a period of consumption. In one embodiment a subjects vitamin A levels increased 50% compared to formulation without mc oil extract. In another embodiment a subject can experience vitamin A level, and other indicator increased significantly after a period of 8 weeks during which the nutritional formulation is consumed once daily in amounts of about 4 g.

The enhanced vitamin A formulation of the present invention can provide for increased absorption of provitamin A carotenoids as compared to an equivalent total vitamin A dosage that does not include mc extract. In one embodiment, the vitamin A absorption is increased by at least about 15% as compared to an equivalent total vitamin A dosage that does not include mc component. In another embodiment the antioxidant absorption can be increased by at least about 25% as compared to an equivalent total vitamin A dosage that does not include mc extract. In yet another embodiment the antioxidant absorption can be increased by at least about 40% as compared to an equivalent total provitamin A dosage that does not include mc extract.

The nutritional formulations of the present invention can be present in a single nutritional composition or in a combination of multiple formulated nutritional compositions which are co-administered to a subject. In one embodiment, the provitamin A formulation is a single liquid formulation containing an effective amount of mc oil extract and at least one carotenoid source.

The nutritional formulations include can include mc extracts and at least one other provitamin A carotenoid source. The at least one other proA carotenoid source can include at least one plant extract. The at least one plant extract can include, but is not limited to mango juice, watermelon juice and papaya juice. Other provitamin A carotenoids well known in the art may also be included. The at least one plant extract can comprise from about 0.1 wt % to about 25 wt % of the nutritional composition. Carotenoids separation were done on a reverse-phase HPLC system following published method (Liquid chromatography-mass spectrometry method for the quantification of bioavailability and bioconversion of beta-carotene to retinol in humans. Wang Y et al. Anal Chem. 2000 Oct. 15; 72(20):4999-5003.).

In one embodiment, the at least one plant extract can include or consist of mango purre. In another embodiment, the at least one fat source can include or consist of avocado fruit or extract and a watermelon juice.

The mc component of the present formulation is generally included as a dried pulp, and may be obtained by any process of processing known to those skilled in the art. By way of example, without limitation, the dried pulp can be done by free-drying, and emulsion in juice, or whey protein, or milk. In other cases, the extract may be oil extract using cold press. One method for extracting mc (Gac) oil is described in U.S. Pat. No. 6,770,585 which is incorporated herein by reference.

In accordance with the present invention, the amount of mc extract contained in the nutritional formulation may be varied according the knowledge of one skilled in the art in order to achieve a particularly desired result. However, the mc component content may be from about 0.1% wt % to about 20 wt % of the formulation. In one aspect, the amount may be from about 3.0 wt % to about 15 wt % of the formulation. In another aspect, the amount may be from about 4.0 wt % to about 9.0 wt % of the formulation.

Mango juice when incorporated in the nutritional formulation, can also enhance the health imparting benefits of the formulation. Provitamin A concentration in mango juice was determined as 0.01 to 0.16 μg mL(−1), using HPLC. Mangoes also provide vitamin C, polyphenols and fructose, which can enhance flavor and aroma of the formulation.

According to one aspect of the present invention, the amount of mango juice contained in the nutritional formulation may be varied depending on the amount of other juice components present and the desired result. When mango puree is included in the nutritional formulation it can comprise from about 0.5 wt % to about 10 wt % of the nutritional composition. In yet another aspect, the amount may be about 1.0 wt % to about 5.0 wt % of the provitamin A composition.

Carrot is well known for providing beta-carotene. It also provides fiber and sweetness to the composition. When mango puree is included in the nutritional formulation it can comprise from about 0.5 wt % to about 10 wt % of the nutritional composition. In yet another aspect, the amount may be about 1.0 wt % to about 5.0 wt % of the provitamin A composition.

Other micronutrients which may be combined with the desired mc extract in the formulation of the present invention include zinc, iron, amino acids, ionic minerals, and naturally occurring anti-oxidants. The amino acids contemplated include: alanine, arginine, carnitine, gamma-aminobutyric acid (GABA), glutamine, glycine, histidine, lysine, methionine, N-acetyl cysteine, ornithine, phenylalanine, taurine, tyrosine, and valine, but are not limited thereto. Additionally, the ionic minerals contemplated by the present invention for inclusion in an embodiment of the formulation include both anions and cations.

Excipients may also be present in the nutritional formulations of the present invention. Excipients can include stabilizers, preservatives, flavoring agents, thickeners, etc. Non-limiting examples of excipients include xanthan gum, sodium benzoate, natural and artificial flavorings, pectin, and the like. The excipients can be present individually or in combinations. It is important that the excipients do not alter or inhibit the provitamin A potency of the nutritional formulations.

The nutritional formulations of the present invention can also include an amount of water. Water can comprise from about 1.0 wt % to about 95 wt % of the nutritional formulation.

The amount of beta-carotene compounds obtained from the fruit juice or extracts may vary from fruit to fruit. Typically, the carotenoid family is responsible for most of the pigments found in plants. Beta-carotene produce the orange pigments found in pumpkins and carrots, mangoes, papaya. Vegetables such as spinach, kales also contain beta-carotene.

In yet another aspect of the present invention, a formulation providing even further enhancement of absorption and bioavailability is provided. The formation provides mc component, and a nutritional fat and protein component (yogurt) and a beta-carotene rich component (mangoes).

Generally, the nutritional formulation may be provided as a liquid oral dosage form. However, in one aspect of the present invention the nutritional formulation may be a variety of oral dosage forms are well known to those of ordinary skill in the art, and specific formulation ingredients may be selected in order to provide a specific result. Typically, the nutritional formulation is formulated in a beverage oral form. Other oral dosage forms may also be implemented with the present formulation. For example, and without limitation, oral dosage forms such as beverages, powders, syrups, elixirs, and suspensions, confections, candies, bars. In a further aspect of the present invention the dosage form may utilize an oil form of the composition of the present invention, where the formulation utilizes other ingredients, such as sweeteners and vicosity.

### MODES FOR CARRYING OUT THE INVENTION

### 1) Clinical Trials:

1.a. Comparison of absorption efficiency of provitamin A carotenene from momordica cochinchinens (mc) with in a female subject.

In this study, plasma response of provitamin A carotene before and after the dose was used an indication of bioavailability. Quantitation of plasma carotenoids was done using published HPLC method.

The clinical trial was a cross-over design showing improvement of absorption of provitamin A beta-carotene from MC Formula1 versus beta-carotene supplement, with a 8-week wash-out period between the two experiments. The subject was a 30-yr old healthy female adult who displayed no clinical symptoms of avitaminosis. At the time of the study, the subject was not pregnant, or lactating and had no problems with lipid malabsorption, or alcoholism. Daily carotenoid and vitamin A intakes were determined by self-maintained food records throughout the two months of the trial. For three consecutive days prior to receiving either supplement, the subject consumed a diet low in carotene and vitamin A. The subject fasted for 10 hours before the first dose was consumed. Immediately before the first dose, a 10 mL of fasting blood sample was drawn. The first meal was the MC Formula1 (120 g), containing 15 mg of provitamin A carotenoid and rice. Carotenoid quantification was done on a reverse-phase HPLC system following published methods (Goodman D. et al., 1966) before the study started. The dose was taken with 1 cup of orange juice. The comparative meal, tested 60 days to make sure there was no residual effect, was comprised of 15 mg of beta-carotene (25,000 IU vitamin A, available at Longs Drug Stores, Walnut Creek, Calif. 94956). The dose was over-the-counter soft-gel capsules taken with the 120 g of rice and also with orange juice. Nutritional contents of MC Formula 1 is provided below.

| Nutrients | |
|---|---|
| Energy (Kcal) | 245 |
| Protein (g) (% energy) | 4.4 |
| CHO (g) (% energy) | 50.9 |
| Fat (% energy) | 2 |
| Provitamin A carotenoid (mg) | 3.5 |
| Total carotenoids (mg) | 5 |
| Thiamin (mg) | 0.2 |
| Riboflavin (mg) | 0.02 |
| Niacin (mg) | 2.22 |
| B6 (mg) | 0.14 |
| Folate (mcg) | 4.50 |
| Vitamin E (mg) | 0.34 |
| Calcium (mg) | 27.8 |
| Iron (mg) | 1.6 |
| Zinc (mg) | 0.69 |

Other meals consumed on that day were devoid of carotene and vitamin A. The same dose was repeated for 6 consecutive days. Serial blood samples were drawn at 4,5,6,7,8,9 and 12 hours after the start of each supplementation.

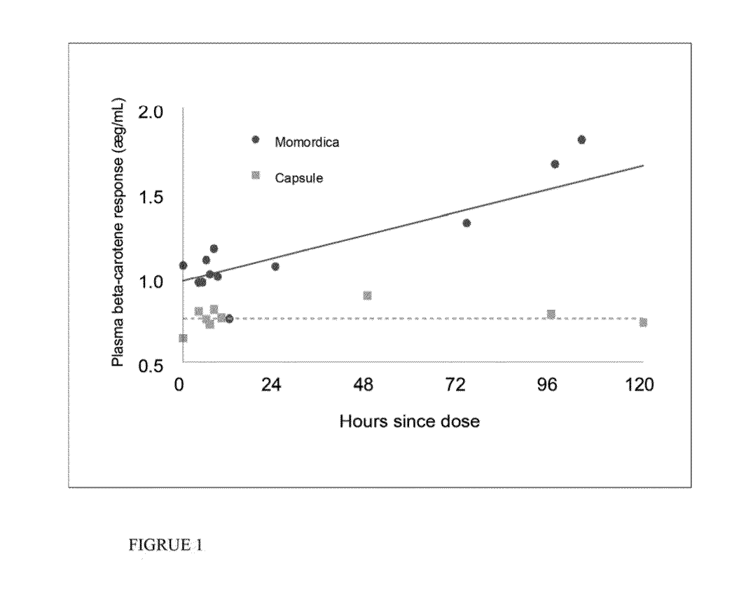

On the 7^{th} day, a final blood sample was taken after 10 hr of fasting. Results of the experiments were shown in FIG. 1.

1.b. This is a large randomized controlled trial. The subjects are pre-school children who had low plasma vitamin A. In the positive control group, the children was given 5 mg of beta-carotene powder supplement with rice; the negative control group receive only rice and the treatment group (MC) receive the MC Formula (momordica cochinchinensis puree and rice protein). All other parameters were the same in the three groups. The table below summaries the changes in plasma provitamin A carotenoids of the MC group before and after the supplementation.

| Plasma levels | Before | After |
|---|---|---|
| Provitamin A carotenoid | 12.62 | 118.74 |
| mcg/dL | | |
| Retinol mcg/dL | 25.70 | 29.79 |

As can be seen from the above clinical trials, absorption efficiency of provitamin A carotenoids was enhanced significantly with the use of momordica cochinchinensis, than convention nutritional supplement.

It is to be understood that the above-described arrangements are only illustrative of the application of the principles of the present invention. Numerous modifications and alternative arrangements may be devised by those skilled in the art without departing from the spirit and scope of the present invention. Thus, while the present invention has been fully described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### 2) Other Examples:

Momordica cochinchinensis powder can be mixed with avocado, mango or in an emulsion containing coconut juice.

In another embodiment, the formulation contains 15 g of MC powder in an emulsion of rice milk.

In another embodiment, the formulation contains 10% of MC powder in an emulsion of yogurt and mango puree.

The above examples will enable those skilled in the art to more clearly understand how to practice the present invention. It is to be understood that while the invention has been described in conjunction with the preferred specific embodiments thereof, that which follows is intended to illustrate and not limit the scope of the invention. Other aspects of the invention will be apparent to those skilled in the art to which the invention pertains.

### REFERENCES

### US Patent Documents

| | | |
|---|---|---|
| 6,770,585 | Vuong | Feb. 5, 2004 |
| 8,007,837 | Mitra, et al. | Aug. 30, 2011 |
| 6,235,721 | Ghosal | May 22, 2001 |
| 8,197,851 | Bos, et al. | Jun. 12, 2012 |
| 7,435,846 | Olvera, et al. | Oct. 14, 2008 |
| 7,141,712 | Choo | Nov. 28, 2006 |
| 8,329,231 | McNeary et. al | Dec. 11, 2012 |
| 6,682,763 | Kuno, et al. | Jan. 27, 2004 |
| 20040115286 | Lee, Kang-Tae, et. al, | Jun. 17, 2004 |
| 7,572,468 | Ishida, et. al, | Aug. 11, 2009 |
| 6,800,292 | Murad | Oct. 5, 2004 |
| 8,318,220 | Koganov | Nov. 27, 2012 |
| 7,122,211 | Jensen, et al. | Oct. 17, 2006 |
| 8,318,215 | Ryngler-Lewensztain, | Nov. 27, 2012 |
| 8,298,548 | Avila, et al. | Oct. 30, 2012 |
| 8,206,721 | Stutz, et al. | Jun. 26, 2012 |
| 8,277,849 | Dillon, et al. | Oct. 2, 2012 |
| 8,221,746 | Isaacs, et al. | Jul. 17, 2012 |
| 6,365,630 | Fisher, et al, | Apr. 2, 2002 |
| 8,304,455 | Dryer et al. | Nov. 6, 2002 |
| 8,216,619 | Giori, et al. | Jul. 10, 2012 |
| 7,557,146 | Sabio Rey | Jul. 7, 2009 |
| 8,067,045 | Smidt, et. al | Nov. 29, 2011 |
| 20090110789 | Mower et al | Apr. 30, 2009 |
| 7,618,662 | Hines, et al | Nov. 17, 2009 |
| 7,592,024 | Ptchelintsev, et al | Sep. 22, 2009 |
| 7,547,456 | Martin et al | Jun. 16, 2009 |
| 5,460,823 | Jansen et al | Oct. 24, 1995 |
| 5,811,609 | Vilstrup et al | Sep. 22, 1998 |
| 6,639,113 | Runge et al | Oct. 28, 2003 |
| 5,827,539 | Gellenback at al | Oct. 27, 1998 |
| 6,132,790 | Schlipalius | Oct. 17, 2000 |
| 5,773,026 | Schlipalius | Jun. 30, 1998 |
| 6,428,816 | Schlipalius et al | Aug. 6, 2002 |
| 5,780,056 | Akamatsu et al | Jul. 4, 1998 |
| 8,221,766 | Dryer et al | Jul. 17, 2012 |
| 7,776,915 | Morariu | Aug. 17, 2010 |
| 8,148,561 | Ansmann et al | Apr. 3, 2012 |
| 7,780,873 | More-Gutierrez, et al | Aug. 24, 2010 |
| 7,531,196 | Kopas et al | May 12, 2009 |
| 8,168,234 | Tripp, et al. | May 1, 2012 |
| 8,158,168 | Kim, et al. | Apr. 17, 2012 |
| 8,442,819 | Tripp, et al. | Mar. 27, 2012 |
| 8,017,147 | Mazed, et al. | Sep. 13, 2011 |
| 7,790,205 | Tripp, et al. | Sep. 7, 2010 |
| 7,781,572 | Bartlett, et al. | Aug. 24, 2010 |
| 7,521,079 | Sakai, et al. | Apr. 21, 2009 |
| 7,431,948 | Tripp, et al | Oct. 7, 2008 |
| 7,270,835 | Tripp, et al. | Sep. 8, 2007 |
| 6,238,707 | Chun, et al. | May 29, 2001 |
| 5,942,233 | Chang | Aug. 24, 1999 |
| 2011014141 | Nakayama, et al. | Jan. 20, 2011 |

### European Patent Documents

| | | |
|---|---|---|
| WO201206706 (A1) | Kreuter, et al., | Feb. 9, 2012 |
| KR20090085844 (A) | Woo, et al. | Aug. 10, 2009 |
| KR200S0026889 (A) | Iotsova, et al. | Mar. 16, 2005 |
| CN1629277 (A) | Feng | Jun. 22, 2005 |
| JP2006169111 (A) | Takashi, et al. | Jun. 29, 2006 |
| CA2281425 (A1) | Gerd, et al. | Mar. 4, 2000 |
| JPH115727 (A) | Yasutomo, et al. | Jan. 12, 1999 |
| FR2760366 (B1) | Person | Sep. 11, 1998 |
| JFH092939 (A) | Yasuhiko, et al, | Jan. 7, 1997 |
| JPS6287519 (A) | Shoichi | Apr. 22, 1987 |

### Other References

Smith, F. R & Goodman, D. S. 1976. Vitamin A transport in Human vitamin A toxicity. N. Engl. J. Med., 294:805-808.
Humphrey, J. H. 1998. Neonatal vitamin A supplementation: effect on development and growh at 3 tof age. Am. J. Clin. Nutr., 68:109-117.
de Francisco, A. 1995. Bulging fontanelle after supplementation with 25000 IU vitamin A in infancy using immunization contacts. Acta Paeditrica, 84:863-866.
Blomhoff, R. 1991. Vitamin A metabolism: new perspectives on absorption, transport and storage. Physiol. Revs., 71:951-990
Vuong, A method of preserving and testing the acceptability of gac fruit oil, a good source of beta-carotene and essential fatty acid, 2003, Food Nutr Bull, 24, 372-3. cited by examiner.
Solomon, in response to Vuong and King, “A method of preserving and testing the acceptability og gac fruit oil, a good source of beta-carotene and essential fatty acids,” 2003, Food and Nutrition Bulletin, 24: 372-374. cited by examiner.
Vuong and King, “A method of preserving and testing the acceptability og gac fruit oil, a good source of beta-carotene and essential fatty acids,” 2003, Food and Nutrition Bulletin, 24: 224-230. cited by examiner.
Le T. Vuong, Stephen R. Dueker, and Suzanne P Murphy, Plasma .beta.-carotene and retinol concentrations of children increase after a 30-d supplementation with the fruit Momordica cochinchinensis (gac). American Society for Clinical Nutrition. 2002. 872-9. cited by other.
Hiromitsu Aoki, Nguyen Thi Minh Kieu, Noriko Kuze, Kazue Tomisaka and Nguyen Van Chuyen, Carotenoid Pigments in GAC Fruit (Mamordica cochinchinensis Spreng), Biosci. Biotechnol. Biochem., 2002, 2479-2482, 66(11). cited by other.
Doug Burke, Genesis Juice, Feb. 1, 2005. cited by other.
Pharmanex introduces G3 nutrition drink, scientifically shown to have high levels of antioxidants. Press Release [online], Pharmanex, Mar. 17, 2005 [retrieved on Oct. 11, 2007]. Retrieved from Internet:, URL:http://www.pharmanex.com/corp/pharmanews/pressreleases/03-1-7-05.shtml. cited by other.
Clinical study: Validation of non-invasive raman spectroscopy as an indicator of total antioxidant nutritional status in humans. Datasheet [online], Pharmanex. October 2005 (retrieved on Oct. 18, 2007), URL: http://download.oneteamglobal.comnus/info.sub.--studies/scanner/ClinicalS-tudy.pdf. cited by other.

What is claimed is:

1. A method of improving absorption and bio-conversion of pro-vitamin A carotenoids by combining at least one beta-carotene containing plant extract with an effective amount of a mc extract.

2. The method in claim 1 wherein the mc extract includes dried powder, concentrate, puree, or oil extract from gac fruit also known as spiny gourd or redmelon, whose scientific name is Momordica cochinchinensis Spreng (mc)

3. The method in claim 1 wherein the pro-vitamin A absorption is increased by at least 15 to 50% as compared to an equivalent total pro-vitamin A dosage that does not include mc extract

4. The method in claim 1 wherein the provitamin A (beta-carotene, alpha-carotene) might be contained a lipid

5. The method in claim 1 wherein the lipid might be selected from edible oil such as “gac” oil, avocado, olives or coconut or palm oil.

6. The method in claim 1 wherein the added lipid may be 1 to 98 wt % of the total wt of the product

7. The method in claim 1 wherein the lipid component also contains vitamin E, a natural preservative, of at least of 2-5 wt %

8. The method in claim 1 wherein the formulation may contain whey protein, soy protein or rice protein

9. The method in claim 1 wherein the added protein may be 10 to 90 wt % of the total wt of the product

10. The method in claim 1 wherein the formulation also contains vitamin E, a natural preservative, of at least of 2-5 wt %

11. The method in claim 6 wherein the mc extract components, lipid, protein and other nutrients are administered as a single composition

12. The method in claim 5 wherein mc extract component comprises from 10 to 40 wt % of total weight of the composition

13. The method in claim 5 wherein >50% of total carotenoids are pro-vitamin A carotenoids

14. The method in claim 6 wherein pro-vitamin A carotenoids (alpha-carotene, beta-carotene) comprise of 60-80 wt % of total carotenoids

15. The method in claim 12 wherein the other plant extract might be selected from the group consisting of mango, carrots, tomatoes, watermelon, pomegranate, dragon fruit, or a mixture thereof

16. The method in claim 13 wherein the other ingredient might be a protein such as yogurt, soy milk, protein drink, rice milk

17. The method in claim 13 wherein the other ingredient might be a, or fat, such as butter, margarine, or natural flavor

18. The method in claim 13 wherein the other ingredient might be a, or extract, or puree, or concentrate, or juice selected from the group of mango, carrots, tomatoes, watermelon, pomegranate, dragon fruit or a mixture thereof

19. the method in claim 13 wherein the nutritional composition contains vitamin C (2-30 wt %), a natural preservative

20. the method of claim 4 wherein the nutritional composition includes excipients selected from the group consisting of flavorants, colorants, stabilizers and preservatives

21. an oral dosage composition for delivering provitamin A carotenoids to a subject comprising: at least one provitaminA carotenoid and mc extract

22. the oral dosage composition of claim 17, wherein the mc extract comprises from 5 to about 20 wt % of the composition

23. the oral dosage composition of claim 18 wherein the mc extract comprises from 10% to 15% of the composition

24. the oral dosage composition of claim 19 wherein the at least one pro-vitamin A carotenoid is present as part of at least one plant extract.
